(19) United States
(12) Patent Application Publication
(10) Pub. No.: US 2019/0151133 A1
(43) Pub. Date: May 23, 2019

(54) ADJUSTING MECHANISM OF SUPPORT BRACE

(71) Applicant: E-LIFE INTERNATIONAL CO., LTD., NEW TAIPEI CITY (TW)

(72) Inventors: Shih-Hsiang LEE, NEW TAIPEI CITY (TW); Yun-Teng Chang, NEW TAIPEI CITY (TW)

(73) Assignee: E-LIFE INTERNATIONAL CO., LTD., NEW TAIPEI CITY (TW)

(21) Appl. No.: 15/820,855

(22) Filed: Nov. 22, 2017

(51) Int. Cl. A61F 5/32; A61F 5/01

(52) U.S. Cl. CPC A61F 5/32; A61F 5/013; A61F 2005/0167; A61F 2005/0158; A61F 5/0123

(57) ABSTRACT

An adjusting mechanism has a longitudinal axis, a lateral axis, a first assembling component, a second assembling component, a worm assembly, a ratchet component, and a knob. The longitudinal axis and the lateral axis are perpendicular to each other. The second assembling component is pivotally mounted to the first assembling component and is able to swing along the longitudinal axis. The worm assembly is mounted to the first assembling component, is able to rotate along the lateral axis, and has a thread twisting along the lateral axis. The ratchet component is mounted to the first assembling component, is able to rotate along the longitudinal axis, is connected to the second assembling component, and has a toothed portion with multiple engaging teeth parallel with the longitudinal axis engaging with the thread of the worm assembly. The knob is mounted to the worm assembly and is rotatable with the worm assembly.

### BACKGROUND OF THE INVENTION

### 1. Field of the Invention

The present invention relates to equipment of orthotics, and more particularly to an adjusting mechanism that is able to be adjusted without specialized tools and is convenient for adjusting.

### 2. Description of Related Art

Elbow support braces, knee support braces, and wrist support braces are common types of equipments of orthotics. They are used to support a human body or even correct musculoskeletal deformities of the human body caused by sports injuries, rheumatoid arthritis, and so on.

A conventional knee support brace has a pivoting mechanism, an upper cuff, and a lower cuff. The pivoting mechanism has a hexagonal socket for accommodating an Allen key. The upper cuff is designed for mounting around the thigh. The upper cuff is connected to the pivoting mechanism and is swingable relative to the pivoting mechanism. The lower cuff is designed for mounting around the calf. The lower cuff is connected to the pivoting mechanism and is swingable relative to the pivoting mechanism. An angle is defined between the upper cuff and the lower cuff both connected to the pivoting mechanism. A wearer may insert the Allen key into the hexagonal socket and swing the Allen key to adjust the angle between the upper cuff and the lower cuff.

However, the knee support brace has to be adjusted with a specialized tool such as the Allen key. That means the wearer needs to prepare the Allen key in advance and is not able to adjust the knee support brace anytime anywhere.

To overcome the shortcomings of the conventional knee support brace, the present invention provides an adjusting mechanism of a support brace to mitigate or obviate the aforementioned problems.

### SUMMARY OF THE INVENTION

The main objective of the present invention is to provide an adjusting mechanism of a supporting brace that able to be adjusted without preparing specialized tools in advance and is convenient for adjusting anytime anywhere.

The adjusting mechanism comprises a longitudinal axis, a lateral axis, a first assembling component, a second assembling component, a worm assembly, a ratchet component, and a knob. The longitudinal axis and the lateral axis are perpendicular to each other. The second assembling component is pivotally mounted to the first assembling component and is able to swing along the longitudinal axis. The worm assembly is mounted to the first assembling component, is able to rotate along the lateral axis, and has a thread twisting along the lateral axis. The ratchet component is mounted to the first assembling component, is able to rotate along the longitudinal axis, is connected to the second assembling component, and has a toothed portion with multiple engaging teeth aligned with the longitudinal axis engaging with the thread of the worm assembly. The knob is mounted to the worm assembly and is able to rotate with the worm assembly.

Other objects, advantages, and novel features of the invention will become more apparent from the following detailed description when taken in conjunction with the accompanying drawings.

### BRIEF DESCRIPTION OF THE DRAWINGS

FIG. 1 is a perspective view of a knee support brace with an adjusting mechanism in accordance with the present invention;

FIG. 2 is an enlarged perspective view of the adjusting mechanism in FIG. 1;

FIG. 3 is an exploded perspective view of the adjusting mechanism in FIG. 2;

FIG. 4 is an operational schematic perspective view of the adjusting mechanism in FIG. 2;

FIG. 5 is another operational schematic perspective view of the adjusting mechanism in FIG. 2;

FIG. 6 is an operational schematic perspective view of the knee support brace in FIG. 1, showing the knee support brace worn on a wearer; and

FIG. 7 is another operational schematic perspective view of the knee support brace in FIG. 1.

### DETAILED DESCRIPTION OF PREFERRED EMBODIMENTS

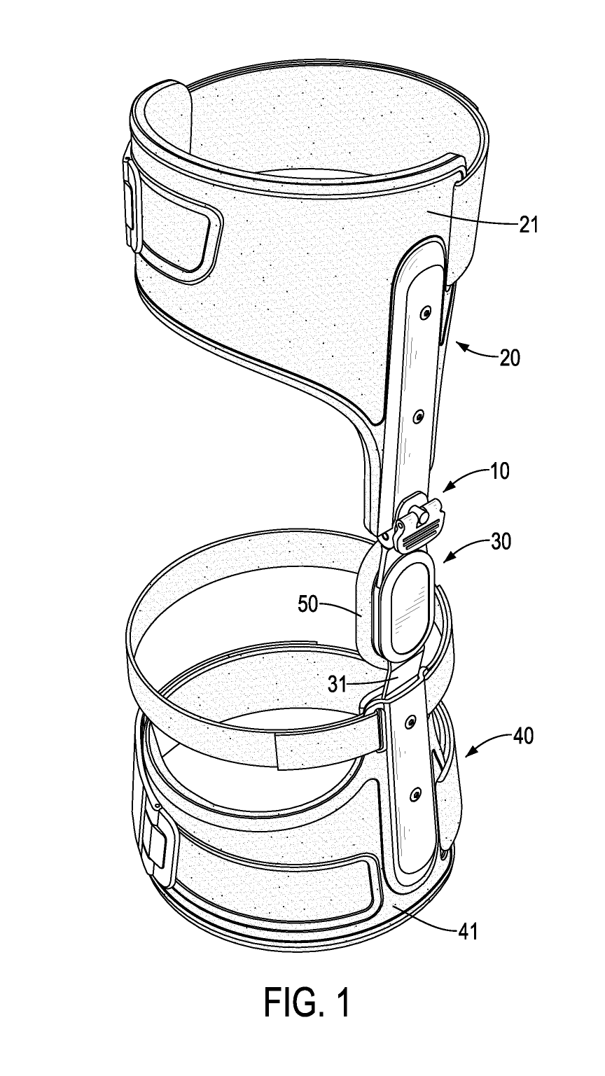

With reference to FIG. 1, a knee support brace with an adjusting mechanism 10 in accordance with the present invention has the adjusting mechanism 10, a first supporting assembly 20, a joint mechanism 30, a second supporting assembly 40, and a lateral pad 50. The first supporting assembly 20 and the joint mechanism 30 are mounted to the adjusting mechanism 10. The second supporting assembly 40 and the lateral pad 50 are mounted to the joint mechanism 30.

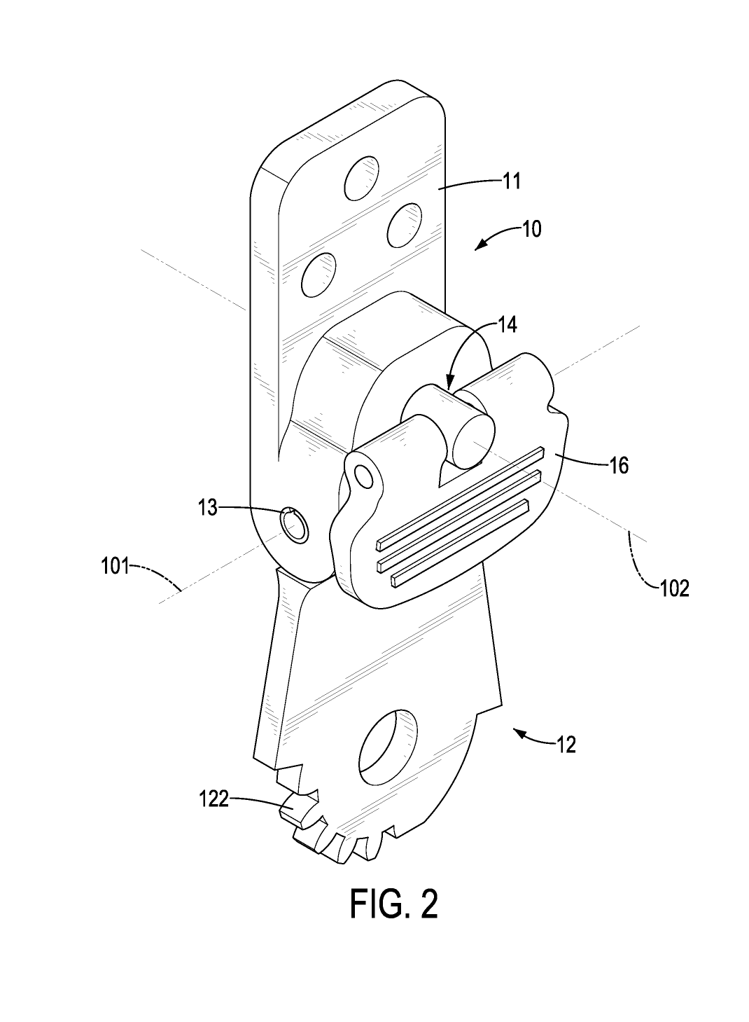

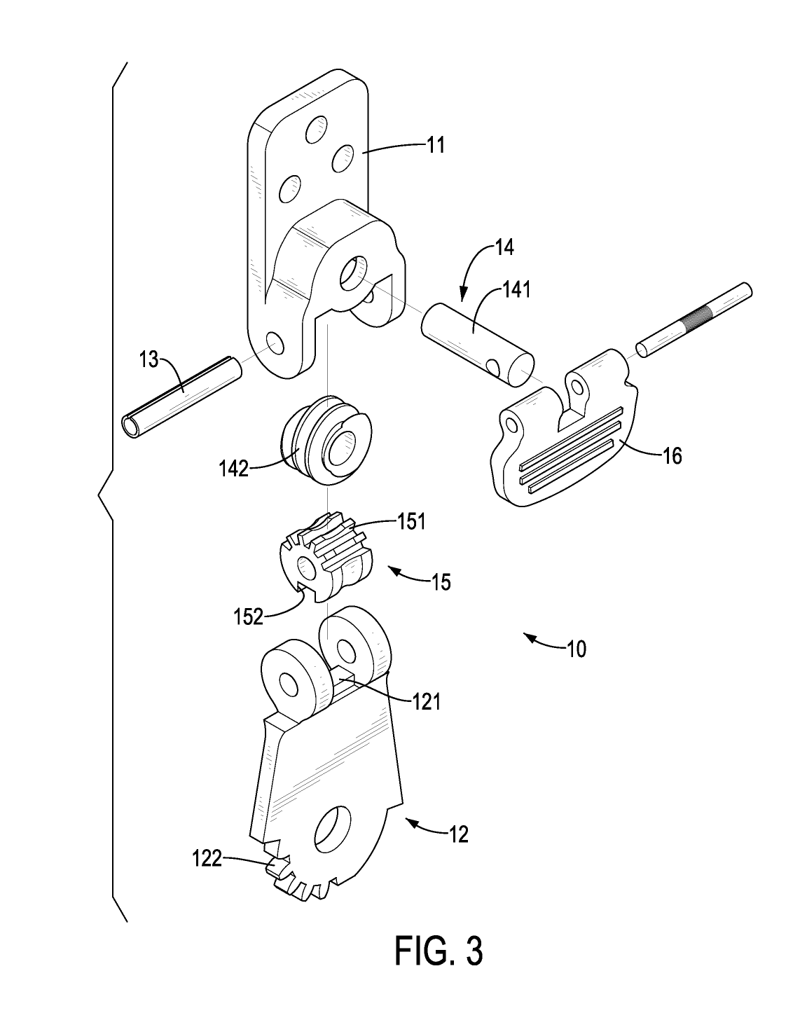

With reference to FIGS. 2 and 3, the adjusting mechanism 10 has a longitudinal axis 101, a lateral axis 102, a first assembling component 11, a second assembling component 12, an assembling shaft 13, a worm assembly 14, a ratchet component 15, and a knob 16. The longitudinal axis 101 and the lateral axis 102 are perpendicular to each other. The first assembling component 11 has two opposite ends. The second assembling component 12 is pivotally mounted to one of the two opposite ends of the first assembling component 11 by the assembling shaft 13 that is disposed along the longitudinal axis 101. The assembling shaft 13 is mounted through the first assembling component 11 and the second assembling component 12, and the first and second assembling components 11, 12 are able to swing relative to each other. The second assembling component 12 has two opposite ends, an engaging block 121, and an engaging portion 122. The engaging block 121 and the engaging portion 122 are respectively disposed at the two opposite ends of the second assembling component 12. The engaging portion 122 has multiple teeth extending along the lateral axis 102.

With reference to FIGS. 2 and 3, the worm assembly 14 is rotatably mounted to the first assembling component 11, is able to rotate along the lateral axis 102, and has a thread twisting along the lateral axis 102. In the embodiment of the present invention, the worm assembly 14 has an operating shaft 141 and a worm gear 142. The operating shaft 141 is rotatably mounted through the first assembling component 11 and is parallel with the lateral axis 102. The worm gear 142 is mounted around and fastened to the operating shaft 141, and is able to rotate with the operating shaft 141. The thread twisting along the lateral axis 102 is formed on the worm gear 142.

With reference to FIGS. 2 and 3, the ratchet component 15 is mounted around the assembling shaft 13 and is mounted to the first assembling component 11 by the assembling shaft 13. The ratchet component 15 is connected to the second assembling component 12 and is able to rotate along the longitudinal axis 101 with the second assembling component 12 that is able to swing relative to the first assembling component 11. The ratchet component 15 has a toothed portion 151 and an engaging recess 152. The toothed portion 151 has multiple engaging teeth extending along the longitudinal axis 101. The toothed portion 151 engages with the thread of the worm assembly 14. In the embodiment of the present invention, the engaging recess 152 and the toothed portion 151 are diametrically opposite each other. The engaging recess 152 is disposed on and engages with the engaging block 121 of the second assembling component 12. Therefore, the ratchet component 15 is able to rotate with the second assembling component 12 that is swingable relative to the first assembling component 11.

With reference to FIGS. 2 and 3, the knob 16 is fastened to the operating shaft 141 of the worm assembly 14 and is able to rotate with the worm assembly 14. In the embodiment of the present invention, the knob 16 is pivotally mounted to the operating shaft 141.

With reference to FIGS. 1 to 3, the first supporting assembly 20 is mounted to one of the two opposite ends of the first assembling component 11 and is distal from the second assembling component 12. The first supporting assembly 20 has a first cuff 21 that is designed for mounting around the thigh of the human body.

With reference to FIGS. 1 to 3, the joint mechanism 30 is mounted to the second assembling component 12 and is able to swing with the second assembling component 12. The joint mechanism 30 has a linkage arm 31 engaging with the engaging portion 122 of the second assembling component 12.

With reference to FIGS. 1 to 3, the second supporting assembly 40 is mounted to the linkage arm 31 and has a second cuff 41. The second cuff 41 is designed for mounting around the calf of the human body.

With reference to FIGS. 1 to 3, the lateral pad 50 is mounted to the joint mechanism 30 and is swingable with the joint mechanism 30 and the second assembling component 12. The lateral pad 50 is designed for pressing a joint of a wearer to provide the joint with lateral force and support the joint of the wearer.

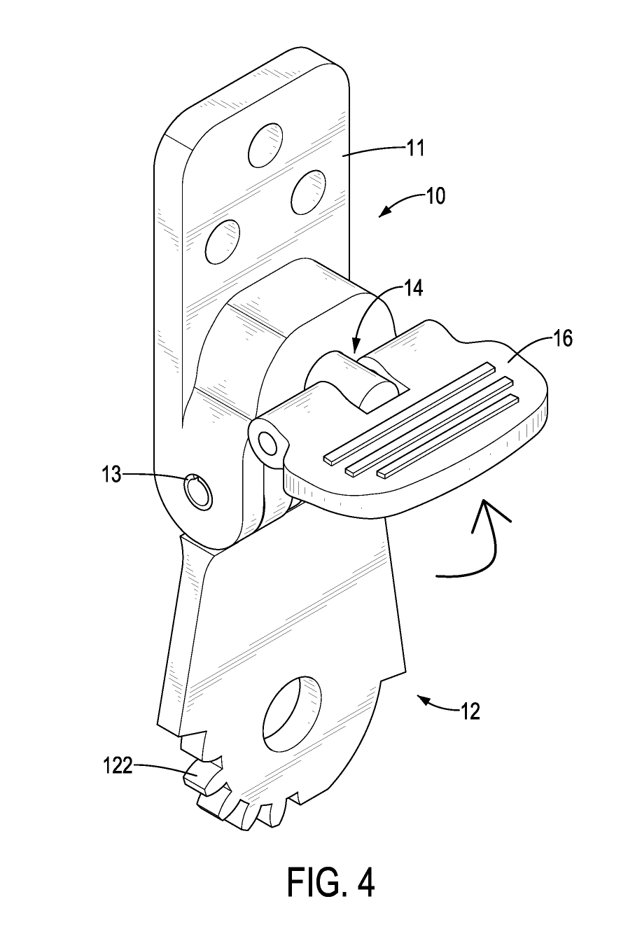

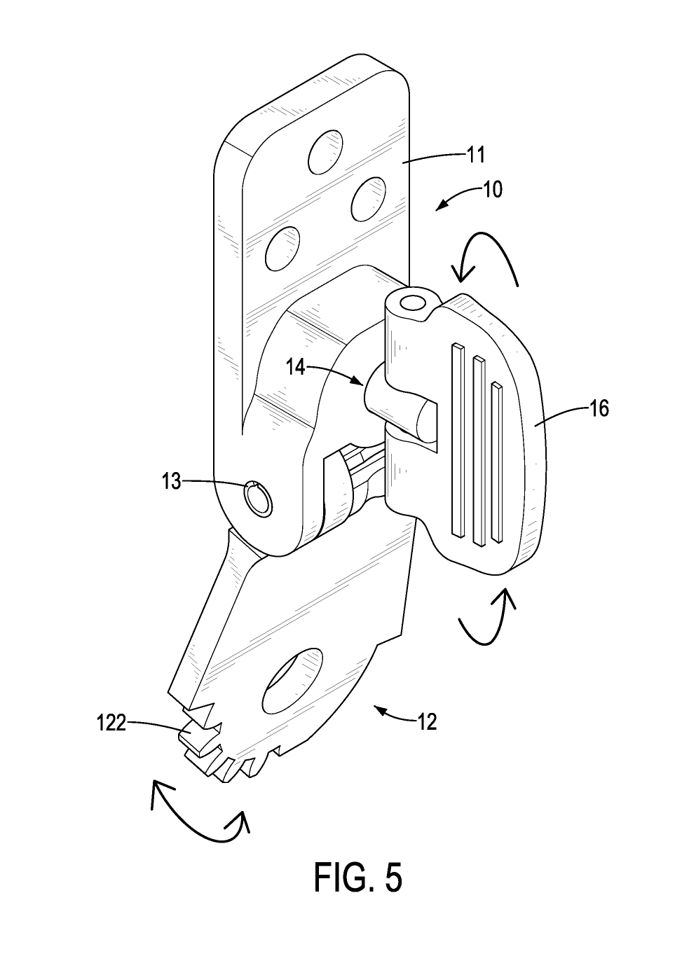

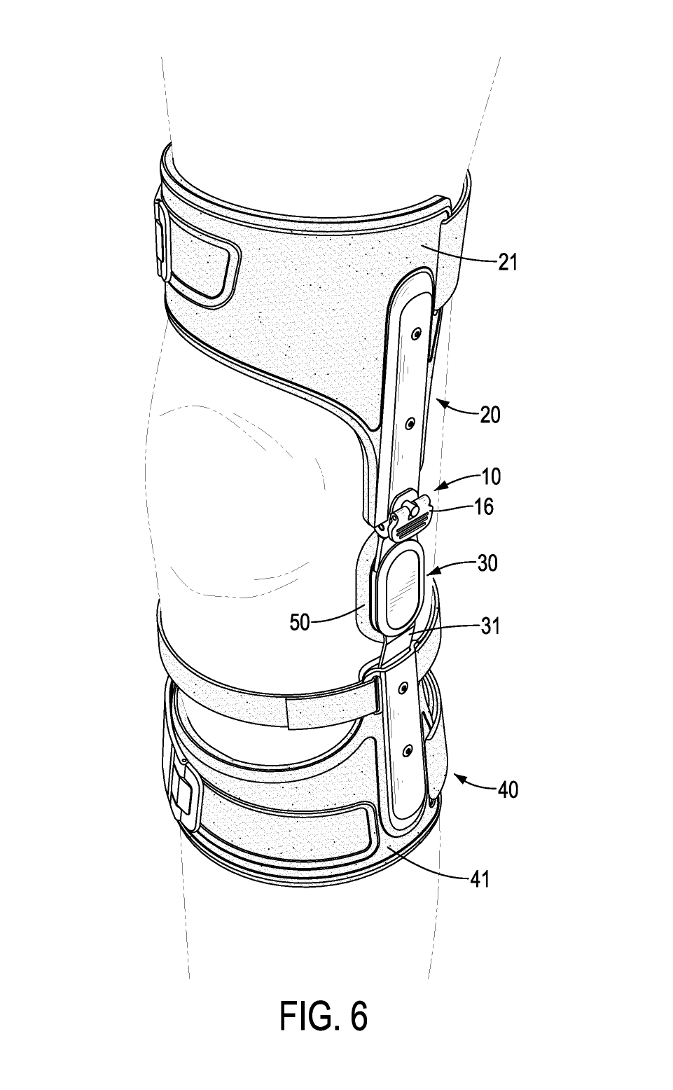

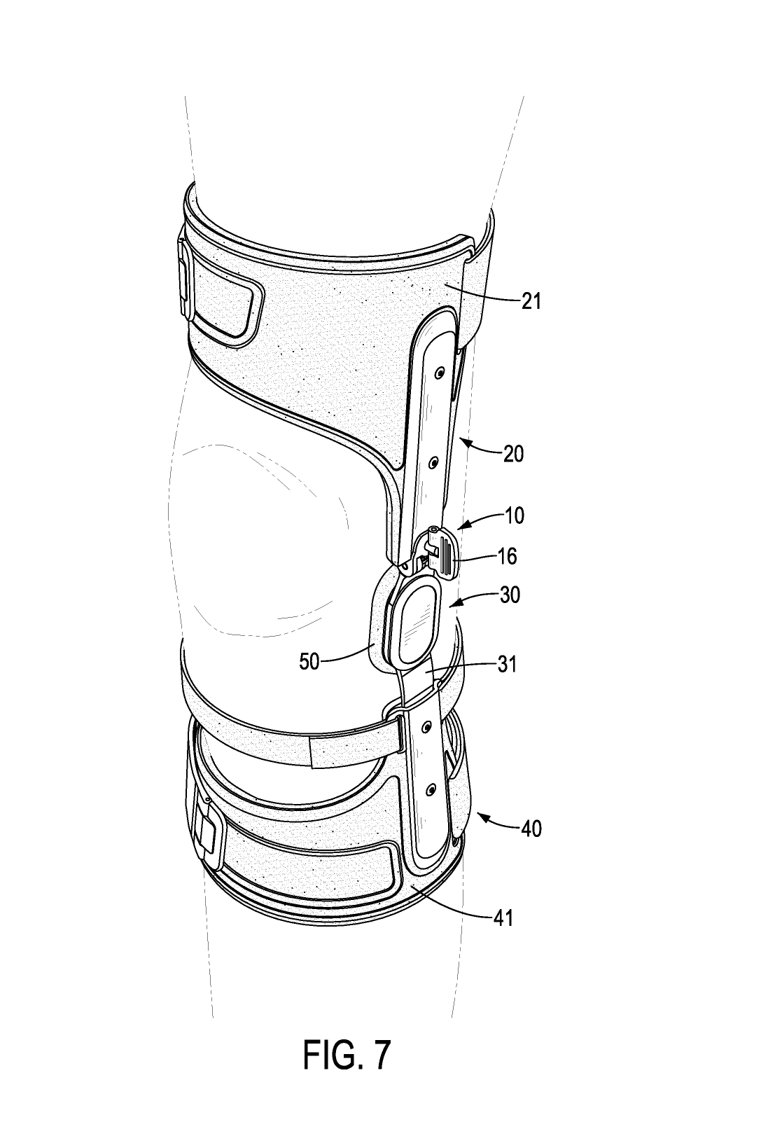

With reference to FIGS. 6 and 7, the first cuff 21 is mounted around a thigh of a wearer, and the second cuff 41 is mounted around a calf of the wearer. With further reference to FIGS. 3 to 5, the knob 16 is turned by the wearer. The knob 16 drives the worm assembly 14 to spin along the lateral axis 102. Since the thread faulted on the worm gear 142 engages with the toothed portion 151 of the ratchet component 15, when the worm assembly 14 spins along the lateral axis 102, the ratchet component 15 is driven by the worm assembly 14 and spins along the longitudinal axis 101. Since the engaging recess 152 of the ratchet component 15 is mounted on the engaging block 121 of the second assembling component 12, the ratchet component 15 and the second assembling component 12 engage with each other.

The second assembling component 12 may spin with the ratchet component 15 and is swingable relative to the first assembling component 11. The joint mechanism 30 mounted to the second assembling component 12 is able to swing together with the second assembling component 12 relative to the first assembling component 11. The lateral pad 50 mounted to the joint mechanism 30 is able to swing with the joint mechanism 30. By operating the adjusting mechanism 10, the wearer is able to adjust the lateral force applied to his/her joint for a better wearing experience. The wearer is able to adjust the knee support brace by directly operating the knob 16 without preparing specialized tools in advance. The adjusting mechanism 10 in accordance with the present invention facilitates the wearer to adjust the knee support brace and provides the wearer with a convenient adjusting means.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. An adjusting mechanism comprising:
a longitudinal axis;
a lateral axis being perpendicular to the longitudinal axis of the adjusting mechanism;
a first assembling component;
a second assembling component pivotally mounted to the first assembling component and being able to swing along the longitudinal axis of the adjusting mechanism;
a worm assembly mounted to the first assembling component, being able to rotate along the lateral axis of the adjusting mechanism, and having a thread twisting along the lateral axis of the adjusting mechanism; and
a ratchet component mounted to the first assembling component, being able to rotate along the longitudinal axis of the adjusting mechanism, connected to the second assembling component, and having a toothed portion with multiple engaging teeth aligned with the longitudinal axis of the adjusting mechanism engaging with the thread of the worm assembly; and
a knob mounted to the worm assembly and being able to rotate with the worm assembly.

2. The adjusting mechanism as claimed in claim 1, wherein the worm assembly has
an operating shaft rotatably mounted through the first assembling component and being parallel with the lateral axis; and
a worm gear mounted around and fastened to the operating shaft, and being able to rotate with the operating shaft.

3. The adjusting mechanism as claimed in claim 2, wherein
the second assembling component has an engaging block; and
the ratchet component has an engaging recess disposed on and engaged with the engaging block.

4. The adjusting mechanism as claimed in claim 3, wherein the engaging recess and the toothed portion of the ratchet component are diametrically opposite each other.

5. The adjusting mechanism as claimed in claim 1, wherein
the second assembling component has an engaging block; and
the ratchet component has an engaging recess disposed on and engaged with the engaging block.

6. The adjusting mechanism as claimed in claim 5, wherein the engaging recess and the toothed portion of the ratchet component are diametrically opposite each other.
